(19) United States
(12) Patent Application Publication
(10) Pub. No.: US 2009/0036664 A1
(43) Pub. Date: Feb. 5, 2009

(54) Complex oligonucleotide primer mix

(76) Inventor: Brian Jon Peter, Los Altos, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/888,388

(22) Filed: Jul. 31, 2007

(51) Int. Cl. C07H 21/04

(52) U.S. Cl. 536/25.3; 536/23.1

(57) ABSTRACT

The invention generally relates to a complex mixture of oligonucleotide primers and/or probes. Another aspect of the invention includes a method of selective priming of a target nucleic acid.

### BACKGROUND

Fluorescence in situ hybridization (FISH) is a cytogenetics technique to fluorescently label and measure specific regions of nucleic acids in cells. FISH can be targeted to either DNA or RNA, and can highlight the prevalence of a specific sequence in a specific cell. For example, FISH probes near chromosomal telomeres can identify small deletions in sub-telomeric DNA, which can be a clinically useful indicator of disease.

Generally, the fluorescent probes for FISH are either prepared using bacterial artificial chromosomes (BACs) or PCR products as template. DNA templates can be labeled using nick translation, using either many random primers (e.g., random hexamers of DNA), or a few specific primers (e.g., primers used to amplify the PCR product). Often the best signal is obtained by looking at repeated sequences, or covering a large region of DNA.

Nick translation is widely used for probe generation in which no exogenous primers are added, but instead single-strand breaks or nicks in the DNA serve as entry sites for a polymerase. As these breaks are usually randomly created by heat, chemical, or DNAse treatment, the resulting labeled probes are still randomly distributed in the template sequences and will suffer from the same issues as probes created from random primers.

Random priming leads to several problems that can reduce signal and create noise in data. For example, if random primers are added, many primers may bind to other primers (“primer dimer”). Also, priming will occur in repeated regions of the nucleic acid, creating probes that will hybridize to other regions of the genome. Regions that are very G/C or A/T rich will also be primed to form probes, but these may not perform as well due to their different Tm's or the propensity to self-hybridize. Many random primers are added in current protocols, but relatively few primers lead to accurate, signal-generating probes, and others contribute to background noise.

Use of only a few specific primers, or random priming of a shorter DNA such as a PCR product, may not provide enough genome coverage to provide a strong signal (especially if the target region is present only in one copy). There is a need for primers and methods that provide coverage of a large region of or a substantial fraction of a genome to produce a strong signal without increasing background noise.

### SUMMARY OF THE INVENTION

The invention generally relates to a complex oligonucleotide mix and methods of generating and using the complex mixture. A plurality of non-random, defined oligonucleotides can be generated on a substrate such as an array. In some embodiments, a composition comprises a plurality of isolated oligonucleotides, at least some of which differ from one another in sequence, length or both. In some embodiments, an oligonucleotide comprises at least two different subsequences when each of the sequences binds to a different site in a target nucleic acid. Oligonucleotides may comprise at least one, two, three, four, or more, cleavage sites. Oligonucleotides can be cleaved from the substrate and/or within the sequence at specific cleavage sites by light, a chemical, or restriction enzymes. Such cleavage can result in oligonucleotides of varying lengths, including, but not limited to, any length from 12 to 250 base pairs (bp), 16 bp, 18 bp, 25 bp, 30 bp, 35 bp, 40 bp, 50 bp, 60 bp, 70 bp, 75 bp, 80 bp, 90 bp, 100 bp, 110 bp, 115 bp, 120 bp, 125 bp, 130 bp, 140 bp, 150 bp, 175 bp, 200 bp, 225 bp, and/or 250 bp. A complex mixture of oligonucleotides can be used as primers and/or probes in assays.

A method of the invention includes preparing a plurality of oligonucleotides, comprising a) selecting at least one target nucleic acid; b) identifying a sequence for each of the plurality of oligonucleotides, wherein each sequence comprises i) a defined sequence of at least 100 nucleotides, wherein at least a portion of the sequence is complementary to a target nucleic acid, and ii) at least one cleavage site; c) synthesizing each of the oligonucleotides at a different address on a substrate; and d) cleaving each of the oligonucleotides from the substrate array. Each oligonucleotide can comprise a different sequence. Alternatively, multiple oligonucleotides can have the same sequence. Methods of the invention provide preparation of a complex mixture of oligonucleotides that can be labeled. Oligonucleotides can be used as primers and probes in well known cytogenetic assays such as fluorescence in situ hybridization (FISH). Using a mixture of complex, defined oligonucleotides allows for a method of selective priming in cytogenetic assays.

### BRIEF DESCRIPTION OF THE FIGURES

FIG. 1 shows the advantages of the selective priming achieved by the methods of the invention and by utilizing the oligonucleotides of the invention. In Random Priming: (A) Two primer sites can directly abut one another; (B) repeat sequences and (C) G/C rich sequences can be primed; and (D) exogenous primers can bind other primers. Selective priming using designed, defined sequences can avoid repeat sequences and G/C rich sequences. Selective priming can also avoid priming a template site that directly abuts a different template site and can avoid primer binding to other primers. Finally, selective priming may be used to differentially prime the 2 strands of DNA.

### DETAILED DESCRIPTION

### Definitions

The terms “nucleic acid,” “nucleotide,” “polynucleotide,” and “oligonucleotide” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. Nucleotides can be produced synthetically, including non-natural, modified nucleotides (e.g., PNA as described in U.S. Pat. No. 5,948,902).

As used herein, the term “defined” refers to a sequence of each distinct oligonucleotide in the plurality that can be predicted with a high degree of confidence. In some embodiments, a defied sequence is a sequence of nucleotides of an oligonucleotide, wherein at least a portion of the oligonucleotide has a sequence designed to bind to a target nucleic acid and includes one or more cleavage sites. In some embodiments, one of the cleavage sites is located at the end of the oligonucleotide to allow for cleavage of the oligonucleotide from the substrate. In some embodiments, the defined oligonucleotide comprises at least two different subsequences separated by at least one of the cleavage site sequences, wherein each of the subsequences of each oligonucleotide binds to a different site in a target nucleic acid or a different target nucleic acid.

As used herein, a “target nucleic acid” refers to a nucleic acid comprising a sequence whose quantity or degree of representation (e.g., copy number) or sequence identity is being assayed. Similarly, “test genomic acids” or a “test genomic sample” refers to genomic nucleic acids comprising sequences whose quantity or degree of representation (e.g., copy number) or sequence identity is being assayed.

The term “primer” refers to an oligonucleotide capable of acting as a point of initiation of synthesis along a complementary strand when conditions are suitable for synthesis of a primer extension product. The synthesizing conditions include the presence of four different deoxyribonucleotide triphosphates and at least one polymerization-inducing agent such as reverse transcriptase or DNA polymerase. These are present in a suitable buffer, which may include constituents which are co-factors or which affect conditions such as pH and the like at various suitable temperatures. A primer is preferably a single strand sequence, such that amplification efficiency is optimized, but double stranded sequences can be utilized.

The term “probe” refers to an oligonucleotide that hybridizes to a target sequence. In some embodiments, a probe includes about eight nucleotides, about 10 nucleotides, about 15 nucleotides, about 20 nucleotides, about 25 nucleotides, about 30 nucleotides, about 40 nucleotides, about 50 nucleotides, about 60 nucleotides, about 70 nucleotides, about 75 nucleotides, about 80 nucleotides, about 90 nucleotides, about 100 nucleotides, about 110 nucleotides, about 115 nucleotides, about 120 nucleotides, about 130 nucleotides, about 140 nucleotides, about 150 nucleotides, about 175 nucleotides, about 187 nucleotides, about 200 nucleotides, about 225 nucleotides, and about 250 nucleotides. A probe can further include a detectable label. Detectable labels include, but are not limited to, a fluorophore (e.g., Texas-Red®, Fluorescein isothiocyanate, etc.,) and a hapten, (e.g., biotin). A detectable label can be covalently attached directly to a probe oligonucleotide, e.g., located at the probe's 5′ end or at the probe's 3′ end. A probe including a fluorophore may also further include a quencher, e.g., Black Hole Quencher™, Iowa Black™, etc.

The term “genome,” as used herein, refers to all nucleic acid sequences (coding and non-coding) and elements present in any virus, single cell (prokaryote or eukaryote) or each cell type in a metazoan organism. The term genome also applies to any naturally occurring or induced variation of these sequences that may be present in a mutant or disease variant of any virus, cell, or cell type. Genomic sequences include, but are not limited to, those involved in the maintenance, replication, segregation, and generation of higher order structures (e.g. folding and compaction of DNA in chromatin and chromosomes), or other functions, as well as all of the coding regions and their corresponding regulatory elements needed to produce and maintain each virus, cell, or cell type in a given organism.

The term “melting temperature” or “Tm” refers to the temperature where the DNA duplex will dissociate and become single stranded. Thus, Tm is an indication of duplex stability.

The terms “hybridize” or “hybridization,” as is known to those of ordinary skill in the art, refer to the binding or duplexing of a nucleic acid molecule to a particular nucleotide sequence under suitable conditions, e.g., under stringent conditions. The term “stringent conditions” (or “stringent hybridization conditions”) as used herein refers to conditions that are compatible to produce binding pairs of nucleic acids, e.g., surface bound and solution phase nucleic acids, of sufficient complementarity to provide for a desired level of specificity in an assay while being less compatible to the formation of binding pairs between binding members of insufficient complementarity to provide for the desired specificity. Stringent conditions are the summation or combination (totality) of both hybridization and wash conditions.

The terms “nucleic acid molecule bound to a surface of a solid support,” “probe bound to a solid support,” “probe immobilized with respect to a surface,” “target bound to a solid support,” or “polynucleotide bound to a solid support” (and similar terms) generally refer to a nucleic acid molecule (e.g., an oligonucleotide or polynucleotide) or a mimetic thereof (e.g., comprising at least one PNA, UNA, and/or LNA monomer) that is immobilized on the surface of a solid substrate, where the substrate can have a variety of configurations, e.g., including, but not limited to, planar substrates, non-planar substrate, a sheet, bead, particle, slide, wafer, web, fiber, tube, capillary, microfluidic channel or reservoir, or other structure. A solid support may be porous or non-porous. In certain embodiments, collections of nucleic acid molecules are present on a surface of the same support, e.g., in the form of an array, which can include at least about two nucleic acid molecules. The two or more nucleic acid molecules may be identical or comprise a different nucleotide base composition.

The term “cleavage site,” refers to a site within a nucleic acid that can be specifically cleaved, e.g., with a restriction enzyme, light, or with certain chemicals. Restriction enzymes (e.g., EcoRI, BlII, BamHI, Sau3A, HindIII, KpnI, etc.) and restriction sites that are recognized by the restriction enzymes are well known. A restriction site for a restriction enzyme can be palindromic. A restriction site may be located within an oligonucleotide in any suitable position. For instance, a restriction site may be located towards one end of the oligonucleotide. An oligonucleotide may contain a hairpin sequence comprising a restriction site, to create a double-stranded restriction site in the oligonucleotide. Alternatively, complement sequences to restriction sites can be added to create double-stranded restriction sites in the oligonucleotides. In certain cases, an oligonucleotide may include more than one cleavage site. A cleavage site typically has a length of 4 nucleotides, 6 nucleotides, or 8 nucleotides, although other lengths are also possible.

The term “CpG island” refers to a sequence of DNA that is greater than 50% G/C and is typically about 200 bp to about 3.0 kb. CpG islands are usually associated with the 5′ ends of numerous genes (Bird, Nature 321: 209-213 (1986)). CpG islands also have an expected/observed G/C ratio of at least 0.6.

For those embodiments where the product plurality is a mixture, the term “mixture” refers to a heterogenous composition of a plurality of different nucleic acids that differ from each other by sequence. Accordingly, the mixtures produced by the subject methods may be viewed as compositions of two or more nucleic acids that are not chemically combined with each other and are capable of being separated, e.g., by using an array of complementary surface immobilized nucleic acids.

The term “substrate” as used herein refers to a surface upon which marker molecules or probes, e.g., an array, may be adhered. Glass slides are the most common substrate for biochips, although fused silica, silicon, plastic, and other materials are also suitable.

The term “address” refers to a predetermined location on a substrate, such as an array. An oligonucleotide at a particular address can be used to detect a particular target or class of targets.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

“Optional” or “optionally,” as used herein, means that the subsequently described circumstance may or may not occur, so that the description includes instances where the circumstance occurs and instances where it does not. For example, the phrase “optionally substituted” means that a non-hydrogen substituent may or may not be present, and, thus, the description includes structures wherein a non-hydrogen substituent is present and structures wherein a non-hydrogen substituent is not present.

### Compositions

In one aspect of the disclosure, a composition is provided that comprises a plurality of isolated oligonucleotides. A composition comprises a plurality of isolated oligonucleotides, wherein at least one or each of the oligonucleotides comprises a) a defined sequence of at least 16 nucleotides, wherein at least a portion of the sequence is complementary to a target nucleic acid, b) at least one cleavage site, and optionally, c) at least two different subsequences separated by at least one of the cleavage site sequences, wherein each of the subsequences of each oligonucleotide binds to a different site in a target nucleic acid or a different target nucleic acid. In some embodiments, the composition comprises oligonucleotides that differ from one another in sequence or length or both. In some embodiments, the composition is useful to provide selective primers for a template such as a BAC or even an entire genome.

A composition includes a plurality of defined, isolated oligonucleotides of at least 100 nucleotides. In some embodiments, the composition comprises defined isolated oligonucleotides, at least some of which are nonidentical in sequence, length or both. A defined oligonucleotide is non-random and has a known sequence. A number of oligonucleotides wherein each sequence is different from one another can be synthesized on the same array. In some embodiments, each oligonucleotide has the same sequence. More than one type of oligonucleotide (i.e., non-identical oligonucleotides) can be used, and each of a plurality of oligonucleotides can bind to various portions of a target nucleic acid (i.e., more than one oligonucleotide may bind to the same portion of the target nucleic acid, and/or to different portions of the target nucleic acid, and/or to combinations thereof).

An oligonucleotide can comprise probes and/or primers that bind to the same or different target nucleic acids. For instance, there may be at least 100 non-identical types of oligonucleotides, at least 1,000 non-identical types of oligonucleotides, at least 10,000 non-identical types of oligonucleotides, or at least 100,000 non-identical types of oligonucleotides. For each type of oligonucleotide, more than one identical molecule of the oligonucleotide may be present in solution. An oligonucleotide may be present in a known or predetermined amount or concentration, or in a known or predetermined ratio, relative to other oligonucleotides. In some embodiments, at least 50% of the oligonucleotides or subsequences thereof bind to a different site in the same target nucleic acid, wherein the target nucleic acid is at least 500 base pairs in length.

Thus, various embodiments of the invention include a composition comprising 2 or more non-identical oligonucleotides, such as 3 or more oligonucleotides, 4 or more oligonucleotides, 5 or more oligonucleotides, 6 or more oligonucleotides, 7 or more oligonucleotides, 10 or more oligonucleotides, 20 or more oligonucleotides, 30 or more oligonucleotides, 40 or more oligonucleotides, 50 or more oligonucleotides, 60 or more oligonucleotides, 70 or more oligonucleotides, 80 or more oligonucleotides, 90 or more oligonucleotides, 100 or more oligonucleotides, 300 or more oligonucleotides, 500 or more oligonucleotides, 1,000 or more oligonucleotides, 3,000 or more oligonucleotides, 5,000 or more oligonucleotides, 10,000 or more oligonucleotides, etc.

Relative amounts and/or concentrations of the different oligonucleotides in the composition may be the same or different. In certain embodiments, a concentration of each different oligonucleotide is known. For example, in some cases, a concentration of each is less than about 10 μM, less than about 5 μM, or less than about 3 μM, less than about 1 μM, less than about 0.75 μM, less than about 0.5 μM, and less than about 0.25 μM. Oligonucleotides may be present in an aqueous fluid, e.g., water, saline, PBS, etc., where the fluid may or may not include further components, e.g., salts, solvents, surfactants, buffers, emulsifiers, chelating agents, etc.

In some embodiments, an oligonucleotide comprises at least two different subsequences separated by at least one cleavage site. As used herein, a first subsequence comprises a contiguous portion of a nucleic acid that is “substantially complementary” or is “able to hybridize” or “binds” to a second, contiguous portion of a target nucleic acid. A nucleic acid that is “substantially complementary” or is “able to hybridize” or “binds” to a second, contiguous portion of a target nucleic acid is one in which at least 75% of the first and second portions are complementary. In some embodiments, the two portions may be at least 80%, 85%, 90%, 95%, 96%, 97%, 98%, 99%, or 100% complementary. In other embodiments, the two portions may include a maximum of 0, 1, 2, 3, 4, 5, 6, 7, 8, 9, or 10 mismatches. First and second portions may be at least substantially complementary for any suitable lengths of each of the two nucleic acids. For example, the two portions of the nucleic acids that are at least substantially complementary may each have complementary portions of at least 50 nucleotides, at least 100 nucleotides, at least 150 nucleotides, at least 200 nucleotides, or at least 250 nucleotides. In some cases, the first and second portions are able to specifically bind to each other (i.e., the nucleic acids exhibit a high degree of specificity); for instance, the first and second portions may be able to bind to each other in a particular configuration or arrangement.

In some embodiments, each oligonucleotide contains more than one subsequence, wherein each subsequence binds to a different site in the target nucleic acid or a different target nucleic acid. In some embodiments, each oligonucleotide comprises at least three different subsequences, each of the subsequences separated by a cleavage site. Each subsequence can have the same or different lengths and can be separated by the same or different cleavage site. In some embodiments, each of the subsequences binds to a different site on the target nucleic acid. In some embodiments, each subsequence or oligonucleotide comprises a primer.

In some embodiments, selective primer sequences are designed to allow flexibility in the size of the region probed. The designed primer sequences can cover any length of a target nucleic acid from a few hundred bp to many Mb. In some embodiments, the primer design may provide non contiguous formats that allow regions of the target nucleic acid to be avoided, such as regions of genomic repeats of duplication.

In some embodiments, the target nucleic acid includes, but is not limited to, genomic DNA, a chromosome, a chromosomal fragment, a bacterial artificial chromosome, a plasmid, and a yeast artificial chromosome or specific regions therein. In some embodiments, the oligonucleotide and/or subsequence are designed to avoid repeat sequences, genomic duplication, and/or CpG sequence in a target nucleic acid. In some embodiments, the oligonucleotide is designed to not bind to a site of a target nucleic acid of at least 200 base pairs comprising at least 50% GC content and an observed or expected CpG ratio of 0.6 or greater. In some embodiments, the oligonucleotide and/or subsequence binds to the sense strand of the target nucleic acid. In other embodiments, the oligonucleotide and/or subsequence binds to the antisense strand of the target nucleic acid. In other embodiments, the oligonucleotide or subsequence thereof binds to a site in the target nucleic acid that is separated by at least 50 base pairs from the site bound by other oligonucleotides or subsequences.

In some embodiments, each of the oligonucleotides or subsequences there of have a Tm for the corresponding target nucleic acid within at least 15° C. of one another. In some other aspects, probes can also be designed to detect a target nucleic acid using duplex Tₘ matching as a design method. In these design methods, candidate probes, with sequences complementary to a target region of interest are identified, and the sequence of the entire target region is searched to find all sequences that can form stable hybrids with the candidate probes (i.e. sequences with homology to the candidate probes). The most homologous sequences are selected, and the candidate probes are modified by deletion or substitution of one or more nucleotides in the candidate probe sequence. The deletion or substitution destabilizes the hybrid pair formed between the candidate probe and the undesired sequences by reducing the Tₘ for the hybrid pairs, below the computed Tₘ of the hybrid between the probe and the desired target sequence. Candidate probes are selected such that (a) the hybrid between the destabilized probe and the desired target is not melted at the chosen assay temperature, and (b) the hybrids between the probe and all undesired homologous targets are melted at the chosen assay temperature, and (c) the melting temperatures of the desired and undesired hybrids are as different as possible. In an aspect, the probes have a Tm difference of about 0.5° C. to about 4° C. when compared to a perfectly matched probe.

Each oligonucleotide may have a suitable length. For example, a length of an oligonucleotide may be at least about 60 nucleotides, 100 nucleotides, at least about 110 nucleotides, at least about 115 nucleotides, at least about 120 nucleotides, at least about 125 nucleotides, at least about 130 nucleotides, at least about 140 nucleotides, at least about 150 nucleotides, at least about 160 nucleotides, at least about 170 nucleotides, at least about 175 nucleotides, at least about 180 nucleotides, at least about 190 nucleotides, at least about 200 nucleotides, at least about 210 nucleotides, at least about 220 nucleotides, at least about 225 nucleotides, at least about 230 nucleotides, at least about 240 nucleotides, or at least about 250 nucleotides. Subsequences of the oligonucleotides may be 12 bp, 16 bp, 18 bp, 25 bp, 30 bp, 35 bp, 40 bp, 50 bp, 60 bp, 70 bp, 75 bp, 80 bp, 90 bp, 100 bp, 110 bp, 115 bp, 120 bp, 125 bp, 130 bp, 140 bp, 150 bp, 175 bp, 200 bp, 225 bp, and/or 250 bp. In some embodiments, primers of about 12 nucleotides or longer are desirable for specific targeting of a particular region of the genome. In some embodiments, primers of 16-18 nucleotides or longer provide for unique positioning within the genome.

Oligonucleotides having such nucleotide lengths may be prepared using any suitable method, for example, using de novo DNA synthesis techniques known to those of ordinary skill in the art, such as solid-phase DNA synthesis techniques, or those techniques disclosed in U.S. Pat. Nos. 6,419,883 and 6,028,189, or Cleary, et al., “Production of Complex Nucleic Acid Libraries using Highly Parallel in situ Oligonucleotide Synthesis,” Nature Methods, 1(3):241-248 (2004). Often, oligonucleotides can be designed with the aid of a computer, based on a sequence of a target nucleic acid and/or a region of interest.

An oligonucleotide of a complex mixture can include one, or more than one cleavage sites. In some embodiments, the oligonucleotide comprises two, three, four, five, six, seven, eight, nine, ten, eleven, twelve, thirteen, fourteen, or fifteen cleavage site(s). A cleavage site can separate at least two different subsequences on the same oligonucleotide. Each oligonucleotide and/or subsequence can be used as a primer or probe. In one step, each oligonucleotide can be cleaved from a substrate and optionally, within the sequence of the oligonucleotide at one or more cleavage sites. When more than one cleavage sequences is present, each cleavage sequence can be the same or different. In an embodiment, when more than one cleavage sequence is present, the cleavage sequences are all the same. In one cleavage step, a mixture of oligonucleotides of varying lengths can be created. A mixture can include oligonucleotides only cleaved from the substrate. A mixture can also include oligonucleotides cleaved into subsequences. Oligonucleotides can be cleaved into at least two subsequences. Oligonucleotides can be cleaved into 2, 3, 4, 5, 6, 7, 8, 9, 10, 11, 12, 13, 14, or 15 subsequences. Thus one cleavage can provide nonrandom, defined oligonucleotides of varying lengths, e.g., 12 bp to 250 bp. Primers or probes can be utilized in assays such as FISH, M-FISH, PCR, Southern blotting.

A cleavage site in an oligonucleotide of the invention can be cleaved by light such as UV light, a chemical (see, e.g., Pon et al., Nucleic Acids Res. 33: 1940-1948 (2005) and Pon et al., Nucleosides Nucleotides Nucleic Acids. 20: 985-989 (2005)), or a restriction enzyme. A cleavage site can be a specific moiety or a specific sequence recognized by restriction enzyme. In some embodiments, at least two of the cleavage sites are cleaved by the same restriction enzyme. In other embodiments, at least two cleavage sequences are cleaved by a different restriction enzyme. In an embodiment, a photocleavable moiety is linked to the oligonucleotide sequence by a spacer or linker. A cleavage site can be a photocleavable moiety such as a phosphoramidite monomer, a DABSYL moiety, 1-(2-nitrophenyl)-ethyl, or an o-nitrobenzyl moiety. Alternatively, oligonucleotides can be cleaved by incubation with chemicals, such as ammonium hydroxide or texaphyrin metal complex (see U.S. Pat. No. 5,798,491). In another embodiment, an oligonucleotide comprises modified nucleotides (see, e.g., Venkatesan et al., Curr. Med. Chem. 10:1973-1991 (2003) such as 7-deaza-7-nitro-dATP, 7-deaza-7-nitro-dGTP, or 5-hydroxy-dCTP, and 5-hydroxy-dUTP separating the different primers. These modified nucleotides can be cleaved by treatment with an oxidant followed by an organic base (Wolfe et al., Proc. Natl. Acad. Sci. USA 99: 11073-11078 (2002)). Restriction enzymes and their recognition sequences are well known. Cleavage by light or by chemical means is advantageous since the oligonucleotide can be cleaved from the substrate and be cleaved itself in the same reaction

In some embodiments, oligonucleotides are synthesized on an array. A plurality of unique oligonucleotides can be synthesized on an array when each oligonucleotide is synthesized at a separate address on the array. Oligonucleotides can be synthesized at defined addresses on an array by several methods, including photochemistry methods that use light to define the polymerization of each nucleotide at each position in the oligonucleotide, or by sequential deposition of reagents such as specific phosphoramidite monomers to react at each position of the oligonucleotide. In some embodiments, the oligonucleotides are labeled. Detectable labels include, but are not limited to a fluorophore, such as Cy3 or Cy5, or a chromogenic moiety or dye, such as an Alexa dye, for example.

In some embodiments, the composition further comprises a polymerase, dNTPs and template DNA. In some embodiments, the oligonucleotides are cleaved from the substrate and optionally into one or more primers or probes to form a composition. The composition comprising the primers is then utilized to synthesize probes with a polymerase, DNTPs, and template DNA in a method, for example, a method of selective priming. The compositions of primers and/or probes as described herein are useful in a variety of methods.

Methods of Preparing and Use of Compositions

A method of the invention includes preparing a plurality of oligonucleotides, comprising a) selecting at least one target nucleic acid; b) identifying a sequence for each of the plurality of oligonucleotides, wherein each sequence comprises i) a defined sequence of at least 100 nucleotides, and ii) at least one cleavage site; c) synthesizing each of the oligonucleotides at a different address on a substrate; and d) cleaving each of the oligonucleotides from the substrate array.

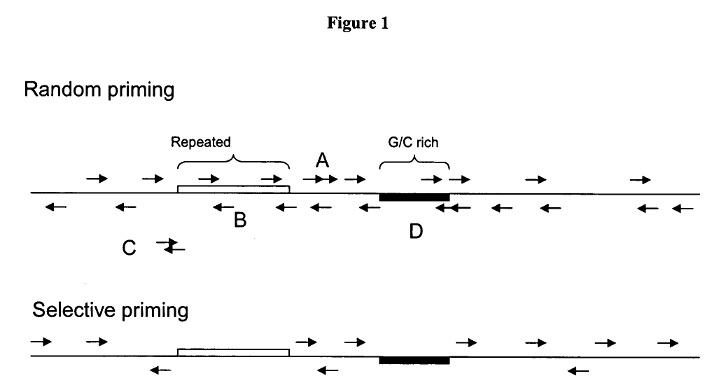

In some embodiments, a plurality of nonrandom, defined oligonucleotides can be designed and/or selected to provide priming at one or more specific location of a target nucleic acid (e.g., a cytogenetic assay such as FISH). Selective priming has several advantages over random priming (see FIG. 1). In selective priming, particular areas or stretches of sequence can be avoided. Selective priming can avoid repeat sequences (e.g., Alu repeats in the human genome) and G/C rich regions (e.g., CpG islands). Selective priming also can prevent “primer dimer” (primers binding to one another), and primers binding to adjacent sites on the target nucleic acid. Selective priming can also result in decreased noise in FISH or other hybridization data due to non-specific hybridization. Additionally, sequences of primers and probes of the invention can be Tm-adjusted to optimize priming specificity.

In an embodiment, a target nucleic acid is selected. In some embodiments, the target nucleic acid includes, but is not limited to, genomic DNA, a chromosome, a chromosomal fragment, a bacterial artificial chromosome, a plasmid, and a yeast artificial chromosome. In some embodiments, the oligonucleotide and/or subsequence are designed based on criteria including, but not limited to, the genomic region probed, the density of primed sites on the genome, the spacing of the primers, and/ or the template strand. In some embodiments, the oligonucleotide and/or subsequence are designed to avoid repeat sequences and/or CpG sequence in a target nucleic acid. In some embodiments, the oligonucleotide is designed to not bind to a site of a target nucleic acid of at least 200 base pairs comprising at least 50% GC content and an observed or expected CpG ratio of 0.6 or greater. In some embodiments, the oligonucleotide and /or subsequence binds to the sense strand of the target nucleic acid. In other embodiments, the oligonucleotide and/or subsequence binds to the antisense strand of the target nucleic acid. In other embodiments, the oligonucleotide or subsequence thereof binds to a site in the target nucleic acid that is separated by at least 50 base pairs from the site bound by other oligonucleotides or subsequences.

Design of selective primer sequences can allow flexibility in the size of the region of a target nucleic acid to be probed. Design for selective priming can allow primer mixes that can span any length, for example, 100 bp to 100 Mb. Primers and/or probes of the invention can also be designed with specific spacing between target sites on the target nucleic acid. Primers and/or probes of the invention can be designed to bind to sites at least about 100 bp, at least about 250 bp, at least about 500 bp, at least about 1.0 kb, at least about 1.5 kb, at least about 2.0 kb, at least about 3.0 kb, at least about 4.0 kb, at least about 5.0 kb, at least about 6.0 kb, at least about 7.0 kb, at least about 8.0 kb, at least about 9.0 kb, at least about 10.0 kb, at least about 100 kb, at least about 500 kb, at least about 750 kb, at least about 1.0 Mb, at least about 2.0 Mb, at least about 3.0 Mb, at least about 4.0 Mb, at least about 5.0 Mb, at least about 6.0 Mb, at least about 7.0 Mb, at least about 8.0 Mb, at least about 9.0 Mb, at least about 10 Mb, at least about 20 Mb, at least about 30 Mb, at least about 40 Mb, at least about 50 Mb, at least about 60 Mb, at least about 70 Mb, at least about 80 Mb, at least about 90 Mb, or at least about 100 Mb between target sites on the target nucleic acid.

In some embodiments, a method comprises identifying a sequence for each of the plurality of oligonucleotides, wherein each sequence comprises i) a defined sequence of at least 100 nucleotides, wherein at least a portion of the sequence is complementary to a target nucleic acid, and ii) at least one cleavage site a sequence. In some embodiments, each oligonucleotide contains more than one subsequence, wherein each subsequence binds to a different site in the target nucleic acid or a different target nucleic acid. In some embodiments, each oligonucleotide comprises at least three different subsequences, each of the subsequences separated by a cleavage site. Each subsequence can have the same or different lengths and can be separated by the same or different cleavage site. In some embodiments, each of the subsequences binds to a different site on the target nucleic acid.

In some embodiments, each of the oligonucleotides or subsequences there of have a Tm for the corresponding target nucleic acid within at least 15° C., 10° C., 5° C., or 1-2° C. of one another. In some other aspects, probes can also be designed to detect a target nucleic acid using duplex Tₘ matching as a design method. In these design methods, candidate probes, with sequences complementary to a target region of interest are identified, and the sequence of the entire target region is searched to find all sequences that can form stable hybrids with the candidate probes (i.e. sequences with homology to the candidate probes). The most homologous sequences are selected, and the candidate probes are modified by deletion or substitution of one or more nucleotides in the candidate probe sequence. The deletion or substitution destabilizes the hybrid pair formed between the candidate probe and the undesired sequences by reducing the Tₘ for the hybrid pairs, below the computed Tₘ of the hybrid between the probe and the desired target sequence. Candidate probes are selected such that (a) the hybrid between the destabilized probe and the desired target is not melted at the chosen assay temperature, and (b) the hybrids between the probe and all undesired homologous targets are melted at the chosen assay temperature, and (c) the melting temperatures of the desired and undesired hybrids are as different as possible. In an aspect, the probes have a Tₘ difference of about 0.5° C. to about 4° C. when compared to a perfectly matched probe.

Each oligonucleotide may have a suitable length. For example, a length of an oligonucleotide may be at least about 60 nucleotides, 100 nucleotides, at least about 110 nucleotides, at least about 115 nucleotides, at least about 120 nucleotides, at least about 125 nucleotides, at least about 130 nucleotides, at least about 140 nucleotides, at least about 150 nucleotides, at least about 160 nucleotides, at least about 170 nucleotides, at least about 175 nucleotides, at least about 180 nucleotides, at least about 190 nucleotides, at least about 200 nucleotides, at least about 210 nucleotides, at least about 220 nucleotides, at least about 225 nucleotides, at least about 230 nucleotides, at least about 240 nucleotides, or at least about 250 nucleotides. Subsequences of the oligonucleotides may be 12 bp, 16, bp, 18 bp, 25 bp, 30 bp, 35 bp, 40 bp, 50 bp, 60 bp, 70 bp, 75 bp, 80 bp, 90 bp, 100 bp, 110 bp, 115 bp, 120 bp, 125 bp, 130 bp, 140 bp, 150 bp, 175 bp, 200 bp, 225 bp, and/or 250 bp.

An oligonucleotide of a complex mixture can include one, or more than one cleavage sites. In some embodiments, the oligonucleotide comprises two, three, four, five, six, seven, eight, nine, ten, eleven, twelve, thirteen, fourteen, or fifteen cleavage site(s). A cleavage site can separate at least two different subsequences on the same oligonucleotide. Each oligonucleotide and subsequence can be used as a primer or probe. In one step, each oligonucleotide can be cleaved from a substrate and optionally, within the sequence of the oligonucleotide at one or more cleavage site. When more than one cleavage sequence is present, each cleavage sequence can be the same or different. In an embodiment, when more than one cleavage sequence is present, the cleavage sequences are all the same. In one cleavage step, a mixture of oligonucleotides of varying lengths can be created. A mixture can include oligonucleotides only cleaved from the substrate. A mixture can also include oligonucleotides cleaved into subsequences. Oligonucleotides can be cleaved into at least two subsequences. Oligonucleotides can be cleaved into 2, 3, 4, 5, 6, 7, 8, 9, 10, 11, 12, 13, 14, or 15 subsequences. Thus one cleavage can provide nonrandom, defined oligonucleotides of varying lengths, e.g., 18 bp to 250 bp. Primers or probes can be utilized in assays such as FISH, M-FISH, PCR, Southern blotting.

A cleavage site in an oligonucleotide of the invention can be cleaved by light such as UV light, a chemical (see, e.g., Pon et al., Nucleic Acids Res. 33: 1940-1948 (2005) and Pon et al., Nucleosides Nucleotides Nucleic Acids. 20: 985-989 (2005)), or a restriction enzyme. A cleavage site can be a specific moiety or a specific sequence recognized by restriction enzyme. In some embodiments, at least two of the cleavage sites are cleaved by the same restriction enzyme. In other embodiments, at least two cleavage sequences are cleaved by a different restriction enzyme.

In an embodiment, a photocleavable moiety is linked to the oligonucleotide sequence by a spacer or linker. A cleavage site can be a photocleavable moiety such as a phosphoramidite monomer, a DABSYL moiety, 1-(2-nitrophenyl)-ethyl, or an o-nitrobenzyl moiety. Alternatively, oligonucleotides can be cleaved by incubation with chemicals, such as ammonium hydroxide or texaphyrin metal complex (see U.S. Pat. No. 5,798,491). In another embodiment, an oligonucleotide comprises modified nucleotides (see, e.g., Venkatesan et al., Curr. Med. Chem. 10:1973-1991 (2003) such as 7-deaza-7-nitro-dATP, 7-deaza-7-nitro-dGTP, or 5-hydroxy-dCTP, and 5-hydroxy-dUTP separating the different primers. These modified nucleotides can be cleaved by treatment with an oxidant followed by an organic base (Wolfe et al., Proc. Natl. Acad. Sci. USA 99: 11073-11078 (2002)). Restriction enzymes and their recognition sequences are well known. Cleavage by light or by chemical means is advantageous since the oligonucleotide can be cleaved from the substrate and be cleaved itself in the same reaction.

In some embodiments, a method comprises synthesizing each of the oligonucleotides on the substrate array. In some embodiments, each oligonucleotide can comprise a different sequence. Alternatively, multiple oligonucleotides can have the same sequence. A plurality of unique oligonucleotides can be synthesized on an array when each oligonucleotide is synthesized at a separate address on the array. Oligonucleotides can be synthesized at defined addresses on an array by several methods, including photochemistry methods that use light to define the polymerization of each nucleotide at each position in the oligonucleotide, or by sequential deposition of reagents such as specific phosphoramidite monomers to react at each position of the oligonucleotide. In an embodiment, 22,000 unique oligonucleotides can be printed. In another embodiment, a thousand oligonucleotides can be printed 22 times each. Embodiments can include a complex mixture of concentrations where some oligonucleotides are printed once, twice, five times, ten times, 50 times, 100 times, 200 times, 250 times, 500 times, 750 times, 1000 times, etc. Methods of the invention provide preparation of a complex mixture of oligonucleotides that can be labeled. In some embodiments, the oligonucleotides are labeled. Detectable labels include, but are not limited to a fluorophore, such as Cy3 or Cy5, or a chromogenic moiety or dye, such as an Alexa dye, or a hapten, such as biotin, for example.

In some embodiments, a method comprises cleaving each of the oligonucleotides from the substrate array. Each of a plurality of subsequences cleaved from an oligonucleotide can be utilized in selective priming methods. An oligonucleotide of the invention can be used as a primer or probe itself without being cleaved within its sequence. For instance a 100 bp can be bound to a substrate and cleaved from the substrate without any cleavage within the 100 bp sequence. Each of a plurality of primers/probes can bind to a target nucleic acid, wherein each primer/probe can bind to the target nucleic acid (i.e., more than one oligonucleotide may bind to the same portion of the target nucleic acid, and/or to different portions of the target nucleic acid, and/or to combinations thereof).

Oligonucleotides can be used as primers and probes in well known cytogenetic assays such as fluorescence in situ hybridization (FISH). Using a mixture of complex, defined oligonucleotides allows for a method of selective priming in cytogenetic assays. Current labeling techniques and kits may be used with the complex mixture of primers and/or probes of the invention, but selective priming can allow for finer spatial targeting of genomic regions, or the generation of probes covering different lengths of the genome. In a complex mixture of primers and/or probes of the invention, there will be enough primers and/or probes in the mixture, that a few poorly binding primers and/or probes will not adversely affect the results. Although array printed oligonucleotides may be produced in small amounts, a gain in sensitivity and signal-to-noise ratio should overcome this limitation.

In some embodiments, the oligonucleotides are cleaved from the substrate and optionally into one or more primers or probes to form a composition. The composition comprising the primers is then utilized to synthesize probes with a polymerase, dNTPs, and template DNA in a method, for example, a method of selective priming. The template DNA comprises genomic DNA, a BAC, a YAC, a chromosome, or a specific region therein. The compositions of primers and/or probes as described herein are useful in a variety of methods.

### Arrays

Embodiments of the invention include arrays, for example, a nucleic acid array. An “array,” includes any one-dimensional, two-dimensional or substantially two-dimensional (as well as a three-dimensional) arrangement of addressable regions bearing a particular chemical moiety or moieties (such as ligands, e.g., biopolymers such as polynucleotide or oligonucleotide sequences (nucleic acids), polypeptides (e.g., proteins), carbohydrates, lipids, etc.) associated with that region. An “addressable region” can be is made up of oligonucleotides bound to a surface of a solid support, also referred to as substrate immobilized nucleic acids. By “immobilized” is meant that a moiety or moieties are stably associated with the substrate surface in the region, such that they do not separate from the region under conditions of using the array, e.g., washing conditions. A moiety or moieties may be covalently or non-covalently bound to the surface in the region. For example, each region may extend into a third dimension in the case where the substrate is porous while not having any substantial third dimension measurement (thickness) in the case where the substrate is non-porous. Arrays of nucleic acids are known in the art, where representative arrays that may be modified to become arrays of the subject invention as described herein, include those described in: U.S. Pat. Nos. 6,656,740; 6,613,893; 6,599,693; 6,589,739; 6,587,579; 6,420,180; 6,387,636; 6,309,875; 6,232,072; 6,221,653; and 6,180,351.

Methods described herein may result in the production of a plurality of nucleic acids, where for each feature present on the template array, there is at least one nucleic acid in the plurality that corresponds to the address. The length of the nucleic acids may be at least about 100 nucleotides, at least about 125 nucleotides, at least about 150 nucleotides, at least about 175 nucleotides, at least about 200 nucleotides, at least about 225 nucleotides, and at least about 250 nucleotides. The plurality of nucleic acids produced in some embodiments may be characterized by having a non-random, defined sequence and/or composition. By “non-random” and/or “defined” is meant that, because of the way in which the plurality is produced, the sequence of each distinct nucleic acid in the product plurality can be predicted with a high degree of confidence. Accordingly, assuming no infidelities, a sequence of each individual or distinct nucleic acid in the product plurality is known. In many embodiments, a relative amount or copy number of each distinct nucleic acid of differing sequence in a plurality is known.

A typical array may contain more than ten, more than one hundred, more than one thousand, more ten thousand addresses, or even more than one hundred thousand addresses, in an area of less than 20 cm² or even less than 10 cm². For example, addresses may have widths (that is, diameter, for a round spot) from about 10 μm to 1.0 cm. In an embodiment, each address may have a width from about 1.0 μm to about 1.0 mm, 5.0 μm to about 500 μm, about 10 μm to about 200 μm, etc. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, or 20% of the total number of features). “Inter-address” areas may be present in some embodiments which do not carry any oligonucleotide (or other biopolymer or chemical moiety of a type of which the features are composed). Such inter-address areas may be present where arrays are formed by processes involving drop deposition of reagents but may not be present when, for example, light directed synthesis fabrication processes are used. It will be appreciated though, that the inter-address areas, when present, could be of various sizes and configurations.

A substrate may have thereon a pattern of addresses (e.g., rows and columns) or may be unpatterned or comprise a random pattern. Addresses may each independently be the same or different.

A substrate may be formed in essentially any shape. A substrate can have at least one surface that is substantially planar. A substrate can also include indentations, protuberances, steps, ridges, terraces, or the like. A substrate may be formed from any suitable material, depending upon the application. For example, a substrate may be a silicon-based chip or a glass slide. Other suitable substrate materials for an array include, but are not limited to, glasses, ceramics, plastics, metals, alloys, carbon, agarose, silica, quartz, cellulose, polyacrylamide, polyamide, polyimide, and gelatin, as well as other polymer supports or other solid-material supports. Polymers that may be used in the substrate include, but are not limited to, polystyrene, poly(tetra)fluoroethylene (PTFE), polyvinylidenedifluoride, polycarbonate, polymethylmethacrylate, polyvinylethylene, polyethyleneimine, polyoxymethylene (POM), polyvinylphenol, polylactides, polymethacrylimide (PMI), polyalkenesulfone (PAS), polypropylene, polyethylene, polyhydroxyethylmethacrylate (HEMA), polydimethylsiloxane, polyacrylamide, polyimide, various block co-polymers, etc.

### Assays

The present invention includes methods of cytogenetic assays. In an embodiment, a cytogenetic assay comprises a composition comprising a plurality of isolated oligonucleotides. In some embodiments, a composition comprises a plurality of isolated oligonucleotides. at least some of which are nonidentical in either sequence, length, or both. Oligonucleotides can be used as primers and/or probes in assays. For instance, an array can be produced with a plurality of oligonucleotides on a substrate surface. There can be 1000 defined, oligonucleotides, 10,000 defined oligonucleotides, or 100,000 defined oligonucleotides, wherein the defined oligonucleotides are not identical to one another. The array can include oligonucleotides of at least 60 bp, 100 bp, 125 bp, 150 bp, 175 bp, 200 bp, 225 bp, and 250 bp. In one reaction, UV light or a chemical can cleave all of the oligonucleotides from the substrate and/or cleave the oligonucleotides into a mixture of oligonucleotides, wherein the oligonucleotides vary in length. A mixture can include the oligonucleotides or subsequences thereof to be utilized as primers or probes of 12 bp, 16 bp, 18 bp, 25 bp, 30 bp, 35 bp, 40 bp, 50 bp, 60 bp, 70 bp, 75 bp, 80 bp, 90 bp, 100 bp, 110 bp, 115 bp, 120 bp, 125 bp, 130 bp, 140 bp, 150 bp, 175 bp, 200 bp, 225 bp, and/or 250 bp. A mixture of oligonucleotides of varying lengths can be collected after the reaction and processed for use in assays. Methods of processing oligonucleotides are well known, and include removal of salts, etc. Methods of processing oligonucleotides can also include labeling the oligonucleotides with a label, e.g., a fluorophore or hapten. Length of a labeled probe can be controlled by using a polymerase without a 5′ exonuclease activity. Labeled probe may be purified away from template nucleic acid.

Cytogenetic assays include, but are not limited to, mapping chromosomes and chromosomal rearrangements, including mapping of an entire genome or chromosome, as well as specific portions or regions of interest in a genome or chromosome. In one aspect, relatively high resolution mapping of chromosomes can be achieved, e.g., resolutions of 1,000,000 bases, 1,000 bases, or even less in some cases. “Resolution” generally refers to regions or segments within the chromosome that can be distinguishably identified. In some embodiments, multiple portions of a genome can be distinguished using well known methods, such as fluorescence in situ hybridization (FISH) or comparative genomic hybridization (CGH) and oligonucleotides described herein. For instance, oligonucleotides may be at least substantially complementary to a chromosome, e.g., substantially complementary to a specific location of a chromosome.

Certain aspects of the invention are directed to systems and methods for mapping genomes or portions of genomes, such as chromosomes. By exposing a genome to a plurality of oligonucleotides that can associate with specific regions of the genome, where at least some of the oligonucleotides are distinguishably labeled, specific regions of the genome can be identified or studied. In some embodiments, regions of less than 1,000,000 bases within a genome can be distinguishably identified, and in some cases, regions of less than 100,000 bp, less than 10,000 bp, less than 1,000 bp, less than 500 bp, less than 300 bp, less than 100 bp, or even less than 50 bp within a genome can be distinguishably identified.

A genome can be from virtually any organism, for example, a human or non-human animal, for example, a mammal such as a dog, a cat, a horse, a donkey, a rabbit, a cow, a pig, a sheep, a goat, a rat, a mouse, a non-human primate (e.g., a monkey, a chimpanzee, a baboon, an ape, a gorilla, etc.); a bird such as a chicken, etc.; a reptile; an amphibian such as a toad or a frog; a fish such as a zebrafish; or the like. A genome can also come from other types of organisms, for example, plants, bacteria, viruses, fungi, molds, yeast, protists, viruses, or the like. A genome may be isolated from a cell, or from tissue, in some cases, as discussed below. An entire genome of an organism may be used in some embodiments. In other embodiments, however, a genome of the organism may be reduced in complexity prior to use. In still other embodiments, only portions of a genome of an organism may be used. For example, in one embodiment, a single chromosome of an organism may be used; in other embodiments, a subset of chromosomes from an organism may be used.

Oligonucleotides of the invention can be used in cytogenetic assays utilizing oligonucleotide primers/probes. Such assays include fluorescence in situ hybridization (FISH), comparative genomic hybridization (CGH), fiber-FISH, multiplex-FISH (M-FISH), primed in situ labeling (PRINS), and polymerase chain reaction. For example, see Wang, Am. J. Med. Genet. 115(3):118-124 (2002) and Dorritie et al., Expert Rev. Mol. Diagn. 4: 663-676 (2004).

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described. All definitions, as defined and used herein, should be understood to control over dictionary definitions, definitions in documents incorporated by reference, and/or ordinary meanings of the defined terms.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention. In this specification and the appended claims, the singular forms “a,” “an” and “the” include plural reference unless the context clearly dictates otherwise.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items.

It should also be understood that, unless clearly indicated to the contrary, in any methods claimed herein that include more than one step or act, the order of the steps or acts of the method is not necessarily limited to the order in which the steps or acts of the method are recited.

All publications mentioned herein are incorporated herein by reference for the purpose of describing and disclosing the invention components that are described in the publications that might be used in connection with the presently described invention.

### EXAMPLES

### Example 1

### Generating Probes for Fluorescence In Situ Hybridization

A method is exemplified for generating probes for Fluorescence In Situ Hybridization. There is interest in creating more specific FISH probes (Mora et al., Mol. Cell Probes 2006, 20: 114-120). The exemplified probes can also be adapted for other cytogenetic techniques, such as Fiber-FISH, Comparative Genomic Hybridization (CGH), Representative Oligonucleotide Microarray Analysis, Primed In Situ Labeling, etc.

### Method

A region of DNA (“the target”) is chosen to probe. In this example, a region of the human genome is chosen. Primers are designed that bind in the target region. The primers avoid highly repeated regions. Primer design strategies and algorithms are well known. These strategies are adapted to design the probe primers for FISH. A complex mixture of primers is synthesized. The primers are synthesized on a glass surface, such as in Agilent's in situ synthesis microarray printing process. The synthesized oligonucleotides are 250 nucleotides in length. The oligonucleotide sequences comprise cleavage sites so that the oligonucleotides can be cleaved from the substrate and cleaved into oligonucleotide fragments in one step. The oligonucleotides are separated from the glass surface using a chemical cleavage (Cleary et al., Nat. Methods 2004, 1: 241-248). The microarray substrate is treated for 2 h with 2-3 ml of 35% ammonium hydroxide at room temperature. The solution is transferred to a 1.5 ml microcentrifuge tube and speed vacuum dried overnight at 45° C. The complex primer mixture includes oligonucleotides of 250 nucleotides, or of long oligonucleotides which are cleaved into several, shorter sequences. Thus the complex mixture includes oligonucleotides in multiples of 25 from 25 to 250 nucleotides. Different primers are also present in different amounts in the mixture. Each primer in the mixture has a unique, nonrandom, known sequence that is designed to maximize specificity and/or affinity of binding to the target, or to minimize binding to nonspecific (off-target) sequences.

The complex mixture of oligonucleotides is used to generate labeled probes targeted to region(s) of interest. Protocols similar to “random priming” are used, substituting the complex primer mixture for the random primers in the protocol. Importantly, the higher specificity of the complex primer mixture allows more complex templates to be used to generate the probes. The more specific complex primer mix creates primers using genomic DNA as a template, without creating labeled primers targeted to highly repeated or problematic regions of the genome. Due to the higher specificity of labeling, a lower concentration of defined primers is used in the labeling mixture compared to protocols using random primers.

### Example 2

### Oligonucleotide Photocleavage

Ink-jet microarrays comprising the oligonucleotides are described herein (see U.S. Pat. Nos. 6,419,883 and 6,028,189). The oligonucleotides are attached to the substrate via photocleavable phosphoramidite monomers. In addition, phosphoramidite monomers separate the primers on the oligonucleotide. Oligonucleotides are cleaved in 1 ml of 25 mM Tris-buffer solution (pH 7.4) by situating the array in direct contact with a UV irradiation source at a wavelength of 302 nm for 20 min.

### Example 3

### DNA Labeling and FISH

This protocol is for use with chromosomal hybridization. Labeling 25-50 ng of template DNA will produce enough probe to detect a single-copy gene on a single slide of chromosome spreads. The total DNA to be labeled can be calculated according to the number of individual slides which are to be probed. To detect repeat sequences, less than 50 ng of probe DNA/slide is necessary to produce a good hybridization signal. The amount required depends on the number of gene copies. For single-copy gene detection, the template should preferably be cosmid or yeast artificial chromosome (YAC) DNA in order to generate sufficient signal; however, templates as small as 15 kb can be used successfully. Miniprep DNA can be used as a template, provided that an RNase step is included in the preparation protocol.

### Labeling Procedure

The reaction buffer is prepared by mixing 8 μl of fluor-12-dUTP with 92 μl of 5× nucleotide buffer in a sterile microcentrifuge tube. In two separate, sterile microcentrifuge tubes, the sample is mixed and control reactions as follows:

### Sample Reaction

50 ng (1-29 μl) of linearized DNA template
0-28 μl of distilled water (dH2O)
10 μl of Defined primer mixture (1100 micrograms/ml in a suitable buffer such as TE)
Total reaction volume is 39 μl

### Control Reaction

50 ng (2 μl) of control DNA template
27 μl of dH2O
10 μl of Defined primer mixture (1100 micrograms/ml in a suitable buffer such as TE)
Total reaction volume is 39 μl

The sample and control reactions are heated to 95-100° C. for 5 minutes in boiling water. The sample and control reaction tubes are centrifuged briefly at room temperature and then placed on ice. The following reagents are added to the sample and control reaction tubes:
10 μl of reaction buffer
1 μl (5 U) of exonuclease-free Klenow
Mix the reaction components with a pipette tip.
Incubate the sample and control reaction tubes at 37° C. for 20-30 minutes.

The reactions are stopped by adding 2 μl of stop mix to each reaction tube. The sample and control reaction tubes are stored at 4° C. in the dark. Unincorporated nucleotides are removed by precipitation:
a. Carrier DNA11 is added to a microcentrifuge tube containing fluorescent probe DNA.
b. 1/10 volume of 3 M sodium acetate (final concentration 0.3 M sodium acetate) is added to the microcentrifuge tube.
c. 1 μg of salmon sperm DNA is added to the appropriate amount of competitor DNA for the hybridization reaction.
The following is a general procedure for performing FISH using the Stratagene Prime-It Fluor Fluorescent Labeling kit.

### Treating the Chromosome Spreads (Day 1)

1. Each slide is spotted with ˜150 μl of RNase in 2×SSC buffer (100 μg/ml). Each slide is covered with a large cover slip and incubate the slides at 37° C. for ˜1 hour. More RNase is added under the edges of the cover slip if necessary. Subsequent washing, dehydration and denaturation steps are performed in Coplin jars.
2. The slides are rinsed briefly in 2×SSC buffer and remove the cover slips.
3. The chromosomes are dehydrated by immersing the slides in a 70, 80 and 100% (v/v) ethanol series at room temperature for 2 minutes each. The slides are allowed to air dry.
4. The chromosomes are denatured by incubating the slides for 34 minutes at 72° C. in a solution of 70% formamide and 2×SSC buffer.
5. The chromosomes are dehydrated by immersing the slides in the following ice-cold ethanol series:
70% (v/v) ethanol for 2 minutes
80% (v/v) ethanol for 2 minutes
100% (v/v) ethanol for 1 minute
6. The slides are immersed in 100% (v/v) ethanol at room temperature for 1 minute and air dry the slides.

### Coprecipitating the Probe DNA

The fluoresceinated probe DNA with 2-10 μg of carrier DNA (e.g., herring sperm DNA) is precipitated with ethanol. If required, 1-5 μg is added to competitor DNA (e.g., total human DNA) or COT-1™ DNA to remove any repeat sequences. The carrier and competitor DNA are sonicated until they are ˜100-500 bp in length.
2. The hybridization buffer is prepared and the buffer is preheated to 42° C. The DNA is suspended in 30 μl of the preheated hybridization buffer.
3. The reaction mixture is incubated at room temperature for 5-10 minutes to ensure that the DNA is well resuspended.

### Denaturing and Preannealing the Probe and Competitor DNA

1. The DNA is incubated for 10-15 minutes at 75° C. The DNA is incubated again at 42° C. for 10-30 minutes if repeat sequences are to be eliminated by annealing with competitor DNA.

### Hybridizing

1. The denatured, annealed DNA is pipeted onto a warmed (37-42° C.) slide of denatured chromosomes.
2. The slide is covered with a glass cover slip (22×30 mm), eliminating as many air bubbles as possible.
3. The edges of the cover slip are sealed with rubber cement and incubated at 37° C. in a humid chamber for 16-24 hours, protected from light.

### Washing the Slides (Day 2)

1. The rubber cement is removed. There is no need to remove the cover slips, because the cover slips will slide off in the first wash step.
2. The slides are immersed as follows:
a. In three washes of 50% formamide and 2×SSC buffer at 45° C. for 5 minutes each with agitation.
b. In three washes of 0.1×SSC buffer at 60° C. for 5 minutes each with agitation.
c. Rinse in 4×SSC buffer and 0.1% Tween®-20 at room temperature.

### Counterstaining the Chromosomes

1. Any residual traces of the rubber cement is removed from the front of each slide. 2.25 μl of antifade [25 mg of triethylenediamine/ml of a 1:1 (v/v) glycerol and phosphate-buffered saline (PBS) solution] containing 200 ng/ml of propidium iodide (PI) and/or 20 ng/ml of 4′,6-diamidino-2-phenylindole (DAPI) is spotted on the slides.
3. Each slide is covered with a glass cover slip (22×30 mm). Any air bubbles are removed from under the cover slip. The slide is turned over and, with the cover slip facing down, the slide is pressed gently to remove the excess antifade. The edges of the cover slip are sealed with clear nail polish.
4. The slides are stored at 4° C. in the dark.
5. The slides are viewed with a fluorescence microscope using a 100× oil immersion objective, a 450- to 490-nm excitation wavelength and a 520-nm emission wavelength filter.

### Example 4

### DNA Labeling Protocol

1. The following is mixed in a clean microcentrifuge tube:
100-1000 ng of template DNA (e.g., YACs, BACs, plasmids, human genomic DNA, etc).
0.1 mM dXTPs,
0.065 mM dTTP,
0.035 mM labeled dTTP (fluorescently labeled, such as fluorescein or Cy3, or hapten labeled, such as biotin, digoxygenin, etc).
20 ug/ml (final concentration) of defined primers
in a volume of 40 μl.
2. The probes are denatured for 15 min at 95° C.
3. 10 μl of SX Klenow buffer (250 mM Tris-HCl pH 7.6, 25 mM MgCl2, 10 mM DTT) is added.
4. 5 units of Klenow enzyme is added and mixed carefully.
5. The reaction is incubated overnight at 37° C.
6. The reaction is stopped by boiling or incubating at 95° C. for 5 min. At this stage the labeled DNA is aliquoted.
7. Unincorporated nucleotides are removed by gel filtration in a spin column (e.g., BioRad's MicroSpin 30), using 1×SSC as buffer.
8. To decrease probe length, the DNA is sonicated with a probe sonicator 12 times for 30 sec with 15 sec cooling intervals on ice. This procedure results in a probe length between 200 and 300 bp.
9. For the hybridization, 600-1000 ng of the labeled probe is mixed and co-precipitated with 10 μg of human cot1 DNA and 100 μg of yeast tRNA as a carrier. The pellet is dissolved in 10 μl of formamide, mixed with 10 μl of 8×SSC, 200 mM Na—PO4 pH 7.0, 0.2% Tween®-20 to reach a final concentration of 50% formamide, 4×SSC, 100 mM Na—PO4 pH 7.0, 0.1% Tween®-20.
10. The DNA probes are denatured by incubating at 95° C. for 5 min before adding the probes to a warmed slide of chromosomal spreads without prior cooling.

We claim:

1. A composition comprising a plurality of isolated oligonucleotides at least some of which are nonidentical in sequence, length or both, wherein each of the oligonucleotides comprises:
a) a defined sequence of at least 16 nucleotides, wherein at least a portion of the sequence is complementary to a target nucleic acid,
b) at least one cleavage site, and optionally
c) at least two different subsequences separated by at least one of the cleavage site sequences, wherein each of the subsequences of each oligonucleotide binds to a different site in the target nucleic acid or each of the subsequences binds to a different target nucleic acid.

2. The composition of claim 1, wherein the target nucleic acid is genomic DNA or a chromosomal fragment.

3. The composition of claim 1, wherein each oligonucleotide or subsequence thereof has a sequence that does not bind to a repeat sequence in the target nucleic acid.

4. The composition of claim 1, wherein each oligonucleotide or subsequence thereof does not bind to a site of the target nucleic acid of at least 200 bp comprising at least 50% GC content and an observed/expected CpG ratio greater than 0.6.

5. The composition of claim 1, wherein each oligonucleotide comprises at least three different subsequences, each of the subsequences separated by at least a cleavage site, wherein each of the subsequences binds to a different site on the target nucleic acid.

6. The composition of claim 1, wherein the cleavage site is specifically cleaved by light, a chemical, or a restriction enzyme.

7. The composition of claim 1, wherein each oligonucleotide comprises at least 100 nucleotides.

8. The composition of claim 1, wherein the oligonucleotides are labelled.

9. A method for preparing a plurality of primers, comprising:
a) selecting at least one target nucleic acid;
b) identifying a sequence for each of a plurality of oligonucleotides, wherein each sequence comprises
i) a defined sequence of at least 100 nucleotides wherein at least a portion of the sequence is complementary to a target nucleic acid, and
ii) at least one cleavage site;
c) synthesizing each of the oligonucleotides at a different address on the substrate; and
d) cleaving each of the oligonucleotides from the array.

10. The method of claim 9, wherein each oligonucleotide is cleaved from the substrate and cleaved into at least two subsequences in step (d).

11. The method of claim 9, wherein the target nucleic acid is genomic DNA.

12. The method of claim 9, wherein each oligonucleotide has a sequence that does not bind to a repeat sequence in the target nucleic acid.

13. The method of claim 9, wherein each oligonucleotide does not bind to a site of the target nucleic acid of at least 200 bp comprising at least 50% GC content and an observed/expected CpG ratio greater than 0.6.

14. The method of claim 9, wherein a cleavage site is a phosphoramidite spacer.

15. The method of claim 9, wherein each oligonucleotide comprises at least three different subsequences, each of the subsequences separated by at least a cleavage site, wherein each of the subsequences binds to a different site on the target nucleic acid.

16. The method of claim 9, wherein the oligonucleotide comprises at least two cleavage sites.

17. The method of claim 9, wherein each oligonucleotide comprises at least 200 nucleotides.

18. The method of claim 9, wherein at least 50% of the oligonucleotides or subsequences thereof bind to a different site in the same target nucleic acid, wherein the target nucleic acid is at least 500 base pairs in length.

19. The method of claim 9 further comprising labeling the oligonucleotides.

20. The method of claim 19, wherein the labeling comprises fluorescent labels.
